(19) United States
(12) Patent Application Publication
(10) Pub. No.: US 2015/0126960 A1
(43) Pub. Date: May 7, 2015

(54) Method for Improved Storage of Suction Catheters

(71) Applicants: Timothy Andrew Rosemore, Reno, NV (US); Donald Lee Rider, Reno, NV (US); Raymond Dee Evans, Reno, NV (US)

(72) Inventors: Timothy Andrew Rosemore, Reno, NV (US); Donald Lee Rider, Reno, NV (US); Raymond Dee Evans, Reno, NV (US)

(21) Appl. No.: 14/522,312

(22) Filed: Oct. 23, 2014

Related U.S. Application Data
(60) Provisional application No. 61/894,761, filed on Oct. 23, 2013.
(60) Provisional application No. 61/915,367, filed on Dec. 12, 2013.

(51) Int. Cl. A61L 2/18; A61B 19/00; A61L 2/20

(52) U.S. Cl. CPC A61L 2/18; A61L 2/202; A61L 2/20; A61B 19/34; A61B 2019/343
USPC 604/500; 422/300

(57) ABSTRACT

A method and system for improved storage of suction catheters are disclosed. The new method and system result in a new suction catheter that has been removed from its packaging prior to use or a used suction catheter being stored in a way that eliminates or reduces the opportunity for cross contamination. The holder also disinfects the suction catheter while it is being stored, thus reducing or eliminating the opportunity for disease or antigens that may be on the new or used suction catheter to infect or reinfect the patient or other individual.

### RELATED APPLICATIONS

This application is based upon and claims the benefit of priority from U.S. Provisional Application No. 61/894,761, filed Oct. 23, 2013, and the U.S. Provisional Application No. 61/915,367, filed Dec. 12, 2013, which application are expressly incorporated by reference herein for all that they teach without exclusion of any portion thereof.

### TECHNICAL FIELD

This disclosure relates generally to the field of medical equipment and, more particularly to a system and method for improving the cleanliness of suction catheter devices.

### BACKGROUND

Methods currently used to store suction catheters result in the opportunity to spread disease or antigens. After a suction catheter is removed from its package, or used, it is stored, typically but not limited to, the area near the patient. It is common practice for the catheter to be hung up, laid near the patient, or placed somewhere it can be used the next time the suction procedure is to be performed. Such methods include for example examples wrapping the suction catheter in an inverter surgical glove, pulling the plastic cover back, rinsing the suction catheter with saline and pulling the plastic cover back down, laying the suction catheter upon the patient's bed (e.g., under the patient's mattress or pillow) and allowing the suction catheter to fall to the floor.

These storage methods allow residual secretions left on or in the suction catheter after the suction procedure is performed to be exposed to the ambient environment. The residual secretions may or may not contain disease agents or antigens. If the residual secretions contain disease or antigens, the disease or antigens can be spread by vectors in the ambient environment. The suction catheter, if not infected with disease or antigens, may be contaminated by disease or antigens from vectors in the ambient environment. Transmission vectors include, but are not limited to, health care workers, the patient or patients, those assisting in the care of the patient, those visiting the area near the patient, equipment used in the care of the patient or the transportation of the patient to another area. There is also an opportunity for disease agents or antigens to grow in or on the suction catheter between uses.

Moreover, the current methods do not prevent cross contamination of a suction catheter after it is removed from its packaging prior to use or after it is used. The current methods also do not minimize or prevent the growth of disease in or on the suction catheter between uses.

The present disclosure is directed, at least in part, to improving or overcoming one or more aspects of the prior art system.

### BRIEF SUMMARY OF THE INVENTION

In a first aspect, the present disclosure describes a new method of storage that eliminates or reduces the opportunity for cross contamination for a new suction catheter that has been removed from its packaging prior to use or a used suction catheter in a way. The holder also disinfects the suction catheter while it is being stored thus reducing or eliminating the opportunity for disease or antigens that may be on the new or used suction catheter to infect or reinfect the patient or patients.

### BRIEF DESCRIPTION OF THE DRAWINGS

The foregoing and other features and advantages of the present disclosure will be more fully understood from the following description of various embodiments, when read together with the accompanying drawings, in which:

FIG. 1 is a simplified perspective view of an embodiment of the disclosed principles showing a holder and associated hardware;

FIG. 2 is a further simplified perspective view of an embodiment of the disclosed principles showing a holder and associated hardware; and

FIG. 3 is a flowchart showing an embodiment of the disclosed principles.

### DETAILED DESCRIPTION

This disclosure generally relates to a safe and effective system and method for suction catheter storage between uses or before use. The system in an embodiment includes a liquid filled disinfectant cartridge, disinfectant cartridge lid, disinfectant cartridge fluid reservoir, disinfectant cartridge guide ribs, disinfectant cartridge lid pull tab, collar, refill port, disinfectant fluid, holder, holder overflow, holder lid, holder overflow sump drain, holder overflow sump drain reservoir, disinfectant cartridge, and holder mount.

The holder can be made using one of or a combination of several types of plastics manufacturing processes. In one embodiment, the liquid filled disinfectant cartridge is formed using a “form, fill, and seal process.” Upon the liquid filled disinfectant cartridge being formed, filled with disinfectant, and then sealed with a lid, the completed liquid filled disinfectant cartridge is placed into the holder that was also formed using a “form, fill, and seal process.” A lid is then placed on the holder. The holder has a holder mount attached to the holder using a bonding method that securely attaches the holder mount to the holder.

It is also contemplated that the liquid filled disinfectant cartridge may be constructed by using a plastic bottle forming process. It is also contemplated that the liquid filled disinfectant cartridge may be constructed using an injection moulding process or extrusion process. It is also contemplated that the liquid filled disinfectant cartridge may be constructed using a blow moulding process. It is also contemplated that the liquid filled disinfectant cartridge may be manufactured using a 3D printer. The liquid filled disinfectant cartridge may be formed using a combination of one or more thermoplastic manufacturing methods, not necessarily mentioned herein.

It is also contemplated that the liquid filled disinfectant cartridge may also be made of a metal or glass construction. It is also contemplated the disinfectant cartridge fluid reservoir may be made from a thin film blowing process wherein the blown thin film disinfectant cartridge fluid reservoir can be attached to an injection moulded or extruded collar. It is also contemplated the holder may be made using one or a combination of the previously mentioned thermoplastic processes. It is also contemplated the holder may be made of a metal or glass construction.

The liquid filled disinfectant cartridge holds the disinfectant fluid. Additionally or alternatively, the disinfectant may be one of, or a combination of, a disinfecting light, a mechanical scrubbing system, a reusable holder and/or a disinfecting gas. As noted above, the holder and liquid filled disinfectant cartridges may be used to provide a method to store and clean other medical devices.

During the course of a patient's treatment, when a suction catheter is removed from its packaging, prior to use or after it is used, there is typically no designated location for the suction catheter to be stored either before it is used or between uses, that would minimize or eliminate the opportunity for contamination of the suction catheter. However, in keeping with the disclosed principles, a person such as a caregiver may use the described holder to store the suction catheter. The care giver first opens the holder lid, if used to seal the top of the holder, and opens a liquid filled disinfectant cartridge by removing the disinfectant cartridge lid. The care giver then turns off the vacuum to the suction catheter and places the suction catheter into the liquid filled disinfectant cartridge.

Upon needing the suction catheter, the care giver turns on the vacuum to the suction catheter, sucking the disinfecting fluid, or other disinfecting mediums, through the suction catheter. This process may be repeated if the holder contained multiple liquid filled disinfectant cartridges or in the case of a holder that contains a refillable (manual or auto-refill) disinfectant. Additionally, the complete holder may be used to store other medical devices.

In order to maximize the efficacy of the method the holder should be located in the patient's ambient environment. The holder minimizes or eliminates cross contamination of the new or used suction catheter. The holder minimizes or eliminates the growth of disease or antigens in or on the suction catheter being stored in the holder. The disinfecting environment in the holder may be accomplished through the use of a holder that is constructed to accomplish disinfecting storage by using a disinfecting method described herein. This method for storing a new or used suction catheter can be used with a patient that is stationary or being moved when the holder is located near the patient.

The holder reduces or eliminates the opportunity for a suction catheter stored using this method from dripping secretions or causing contact transfer into the patient's ambient environment. Otherwise, these secretions can be carried by vectors to other areas, other patients, or other people through cross contamination. If the new or used suction catheter is stored using the present method, the stored suction catheter's exposure to contamination from the patient's ambient environment or by vectors entering the patient's ambient environment is minimized or eliminated.

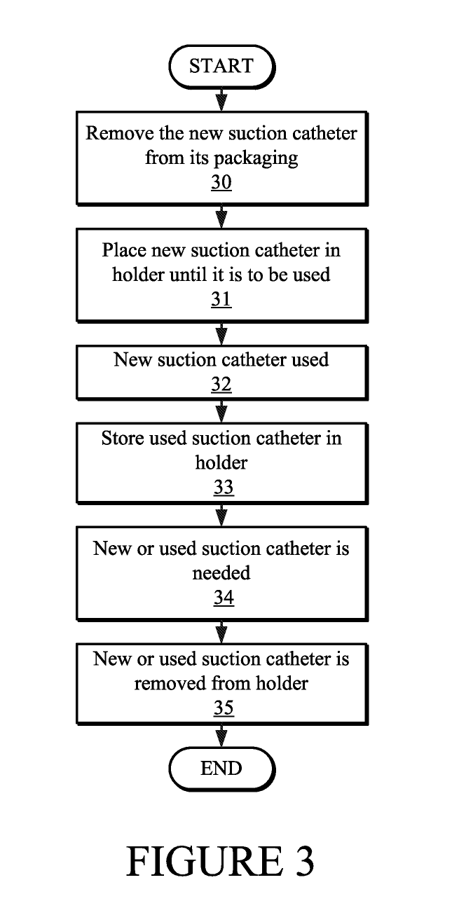

A method in keeping with the disclosed principles is discussed in FIG. 3, in the context of storing a newly un-packaged and yet unused suction catheter or used suction catheter in a holder. The using person removes the new suction catheter from its packaging at stage 30. If the new suction catheter is not immediately needed for use it is then placed in the disinfecting holder at stage 31 until it is to be used. Once the suction catheter is used at stage 32, the used suction catheter is stored in the holder at stage 33. When the new or used suction catheter is needed at stage 34 it is removed from the holder at stage 35 and used. After each use it is placed back in the disinfecting holder. It is contemplated that the disinfecting holder may have reusable or single use storage compartments.

It is contemplated that the holder can be secured to equipment or furniture located near the patient or may sit free. It is contemplated that the holder may have a rigid, semi-rigid, or flexible pouch where the suction catheter would rest. It is contemplated that the holder may be filled with a liquid disinfectant, such as but not limited to, Chlorhexidine. The holder may utilize a flexible film plastic as a pouch to hold the disinfecting fluid such that it will collapse around the suction catheter and invert itself as the suction catheter is removed from the holder prior to use. This further minimizes or prevents contamination of the suction catheter by disease and/or antigens that might have transferred to the guide(s) or upper part of the holder when the suction catheter is inserted into the holder. The inverted reservoir also informs the user that the disinfectant cartridge is spent and cannot be used again,

It is also contemplated that the holder may use a gas, such as but not limited to, ozone to act as a disinfectant. The ozone may be generated in the holder or external to the holder and pumped into the holder. The suction catheter upon being placed into the holder is immersed in the disinfecting gas. Prior to the suction catheter being removed from the holder, applied suction vacuums the disinfecting gas into the suction catheter, down the hose connecting the suction catheter to the collection canister.

It is also contemplated that the holder may use a type of disinfecting light located in the holder, such as but not limited to, ultraviolet light. The suction catheter, upon being placed in the holder is exposed to the disinfecting light in the holder. This disinfects the outside of the suction catheter. If the suction catheter is made of a clear material, the disinfecting light may also disinfect the inside of the suction catheter.

It is also contemplated that the holder may utilize a mechanical method such as but not limited to a scrubbing system to clean the suction catheter that is placed in the holder. This can be achieved by but not limited to a spinning or oscillating brush. It is also contemplated that the holder may possess a reservoir of liquid disinfectant that may be manually operated to refill the disinfectant cartridge fluid reservoir or may auto-refill the disinfectant cartridge fluid reservoir where the suction catheter is placed.

It is also contemplated that the holder may reuse and/or refill the disinfectant cartridge fluid reservoir from an external source of disinfecting fluid. It is contemplated that if there are viscous secretions on the exterior surface of a suction catheter, these viscous secretions may need to be wiped off or removed prior to inserting the suction catheter into the holder. It is contemplated that prior to a suction catheter being inserted into the holder, the suction may be turned off to prevent the suction catheter from drawing the disinfectant gas and/or liquid out of the pouch prior to the suction catheter being fully inserted into the holder. Moreover, turning off the vacuum to the suction catheter prior to being installed in the holder may reduce the overall vacuum system demand. It is contemplated that the holder may have one or more disinfectant cartridges.

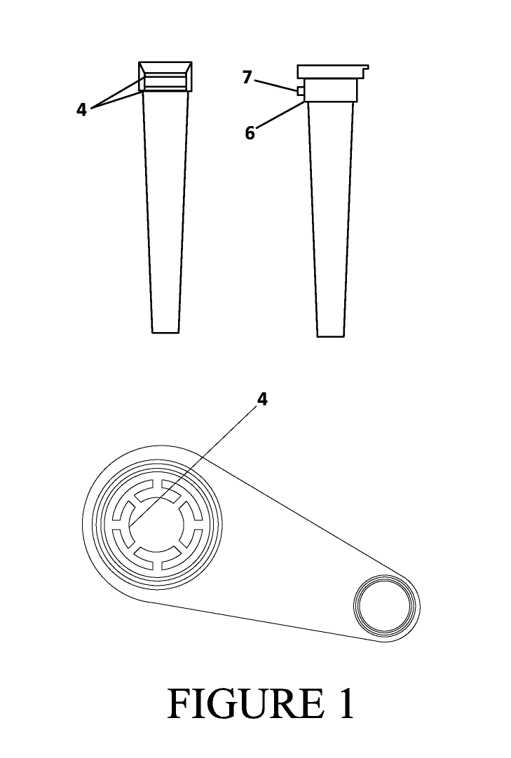

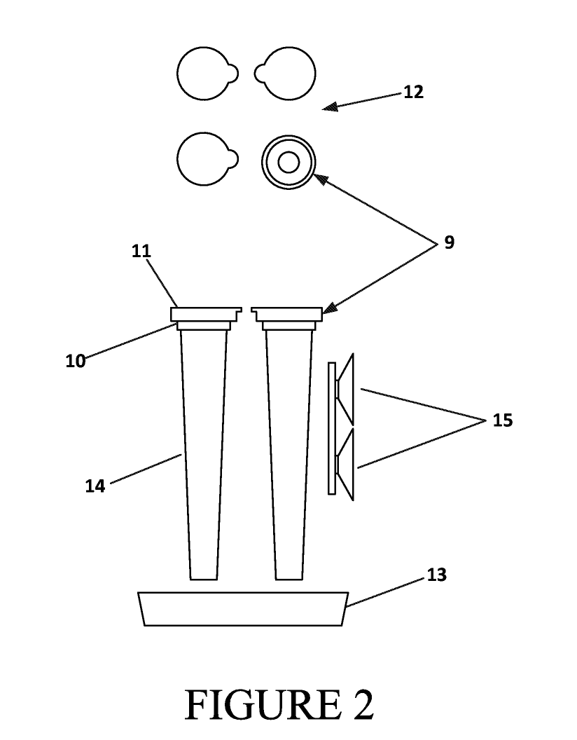

The elements shown in FIGS. 1 and 2 are explained by reference number in the following:

#1. Liquid Filled Disinfectant Cartridge—the liquid filled disinfectant cartridge (1), in this embodiment, is an insert that is loaded or placed into the holder (9). It is also contemplated the liquid filled disinfectant cartridge (1) may be moulded or integrated into the holder (9) as well. the li_q11id filled disinfectant cartridge (1) is made up of but not limited a collar (6), disinfectant cartridge guide ribs (5), disinfectant cartridge fluid reservoir (3), a refill port (7), and disinfectant fluid (8).

#2. Disinfectant Cartridge Lid—The disinfectant cartridge lid (2) shown is a reusable plastic lid that seals the disinfectant into the disinfectant cartridge (1). It is removed prior to the disinfectant cartridge (1) being used to store a suction catheter. It is also contemplated that the disinfectant cartridge lid (2) may be made of a single use foil and/or thin film construction that adheres and seals the disinfectant into the disinfectant cartridge (1).

#3 Disinfectant Cartridge Fluid Reservoir—The disinfectant cartridge fluid reservoir (3) in this embodiment is made of a flexible film plastic. It is contemplated the disinfectant cartridge fluid reservoir (3) may also be constructed of a semi-flexible or rigid material. The reservoir contains the disinfectant fluid (8), It is also contemplated that the diameter and depth and shape of the disinfectant cartridge fluid reservoir (3) may be adjusted to better address different diameters, depths and constructions of different suction catheters.

#4 Disinfectant Cartridge Guide Ribs—disinfectant cartridge guide ribs (4) are located in the liquid filled disinfectant cartridge (1) so that they are submerged in the disinfectant fluid (8). This allows for any contamination by disease and/or antigens located on a new and yet unused suction catheter or used suction catheter that may be scraped off by the passing the suction catheter through the disinfectant cartridge guide ribs (4) to be exposed to the disinfectant fluid (8). The disinfectant cartridge guide ribs (4) position the suction catheter in the center of the liquid filled disinfectant cartridge (1). This minimizes the opportunity for contamination on the suction catheter to be transferred to the top lip of tile liquid filled disinfectant cartridge (1). By centering the suction catheter in the liquid filled disinfectant cartridge (1) using the disinfectant cartridge guide ribs (4) the opportunity is minimized for cross contamination from the lip of the liquid filled disinfectant cartridge (1) to the suction catheter when the suction catheter is removed from the liquid filled disinfectant cartridge (1).

#5 Disinfectant Cartridge Lid Pull Tab—The Disinfectant Cartridge Lid Pull Tab (5), may be a part of the Disinfectant Cartridge Lid (2) construction, enables a person to more easily remove the Disinfectant Cartridge Lid (2) from the Liquid Filled Disinfectant Cartridge (1).

#6 Collar—It is contemplated that the Collar (6) may be rigid or semi-rigid. It is also contemplated the collar may have a Refill Port (7) attached to it or molded and integrated into it. The Collar {6) may also be integrated into the Holder (9) by mechanically fixing to or molding the Collar (6) as part of the Holder (9). TI1e Collar (6) may also be constructed in a way that allows for the suction catheter to be secured mechanically to the Collar (6).

#7 Refill Port—The refill port (7) in this embodiment is a port that uses a lock system. It is also contemplated to use any mechanical port that allows the fluid to flow through the refill port (7) into the liquid filled disinfectant cartridge (1) and that seals the fluid into the liquid filled disinfectant cartridge (1).

#8 Disinfectant Fluid—In this embodiment the disinfectant fluid (8) is a chemical antiseptic solution of chlorhexidine. It is contemplated that any acceptable chemical antiseptic may be used to fill the disinfectant cartridge fluid reservoir.

#9 Holder—The holder (9) includes the liquid filled disinfectant cartridge(s) (1).

#10 Holder Overflow Sump—As a suction catheter is inserted into liquid filled disinfectant cartridge (1) some of the disinfectant fluid (8) may be displaced. The displaced disinfectant fluid (8) may overflow the top of the liquid filled disinfectant cartridge (1). The displaced disinfectant fluid (8) flows into the holder overflow sump (10) where it can flow through the holder overflow sump drain (12) into the holder overflow sump drain reservoir (13). It is also contemplated that the liquid filled disinfectant cartridge(s) (1) might have an overflow port where displaced or spilled disinfectant fluid (8) may flow into the holder overflow sump (10) or directly into the holder overflow sump drain reservoir (13).

#11 Holder Lid—It is contemplated the holder (9) may or may not have a lid. In this embodiment, the holder lid (11) may be a reusable plastic lid that seals the disinfectant cartridge(s) (1) into the holder (9). It is removed prior to the holder (9) being used to store a suction catheter. It is also contemplated that the disinfectant cartridge lid (2) may be made of a single use foil and/or thin 111m construction that adheres and seals the holder (9).

#12 Holder Overflow Sump Drain

#13 Holder Overflow Sump Drain Reservoir—The holder overflow sump drain reservoir (13) may hold the overflowed or spilled disinfectant fluid (8) from the liquid filled disinfectant cartridge(s) (1). It is contemplated that the holder (9) might have a port used to drain the holder overflow sump drain reservoir (13). The port may use a lock system or any mechanical port that allows the fluid to flow out of the holder overflow sump drain reservoir (13) and then seals to minimize spillage from the holder overflow sump drain reservoir (13).

#14 Disinfectant Cartridge

#15 Holder Mount—In this embodiment the holder can be secured using suction cups. It is also contemplated that any mechanical system may be used to secure the holder (9) to anything practical and convenient in the patient's ambient environment.

One skilled in the art will realise the disclosure may be embodied in other specific forms without departing from the disclosure or beneficial characteristics thereof. The foregoing embodiments are therefore to be considered in all respects illustrative rather than limiting of the disclosure herein. The scope of the invention is thus indicated by the appended claims, rather than the foregoing description, and all changes that come within the meaning and range of equivalence of the claims are therefore intended to be embraced therein.

What is claimed is:

1. A system of holding a suction catheter while the suction catheter is not in use, the system comprising:
a holder;
a liquid filled disinfectant cartridge configured to be located in the holder and including a collar, disinfectant cartridge guide ribs, a disinfectant cartridge fluid reservoir, a refill port, and disinfectant fluid;
a disinfectant cartridge lid that seals the disinfectant into the disinfectant and that is removable prior to the disinfectant cartridge used to store a suction catheter; and
a disinfectant cartridge fluid reservoir containing the disinfectant fluid.

2. The system in accordance with claim 1, wherein the liquid filled disinfectant cartridge is an integral part of the holder.

3. The system in accordance with claim 1, wherein the liquid filled disinfectant cartridge is separate from the holder.

4. The system in accordance with claim 1, wherein the disinfectant cartridge lid is a reusable plastic lid.

5. The system in accordance with claim 1, wherein the disinfectant cartridge lid is a single use piece.

6. The system in accordance with claim 5, wherein the disinfectant cartridge lid comprises one of foil and thin film that adheres and seals the disinfectant into the disinfectant cartridge.

7. The system in accordance with claim 1, wherein the disinfectant cartridge fluid reservoir is made of a flexible film plastic.

8. The system in accordance with claim 1, wherein the disinfectant cartridge fluid reservoir is made of a semi-flexible or rigid material.

9. The system in accordance with claim 1, wherein the disinfectant cartridge guide ribs are located in the liquid filled disinfectant cartridge so that they are submerged in the disinfectant fluid.

10. The system in accordance with claim 1, wherein the disinfectant cartridge guide ribs are located to position the suction catheter in the center of the liquid filled disinfectant cartridge.

11. The system in accordance with claim 1, further comprising a disinfectant cartridge lid pull tab.

12. The system in accordance with claim 11, wherein the disinfectant cartridge lid pull tab is integrated into the disinfectant cartridge lid.

13. The system in accordance with claim 1, wherein the disinfectant fluid is a chemical antiseptic solution.

14. The system in accordance with claim 13, wherein the disinfectant fluid comprises chlorhexidine.

15. The system in accordance with claim 1, wherein the disinfectant fluid is a gas.

16. The system in accordance with claim 15, wherein the gas comprises ozone gas.

17. The system in accordance with claim 1, further comprising a holder mount.

18. The system in accordance with claim 17, wherein the holder mount comprises one or more suction cups.

19. A method for maintaining a suction catheter in sterile condition comprising:
placing the suction catheter in a disinfectant cartridge within a cartridge holder, the disinfectant cartridge containing a disinfecting agent;
withdrawing the suction catheter from the disinfectant cartridge; and
using the suction catheter to suction a patient.
